(19) United States
(12) Patent Application Publication
(10) Pub. No.: US 2013/0271728 A1
(43) Pub. Date: Oct. 17, 2013

(54) MULTIPLE-LENS RETINAL IMAGING DEVICE AND METHODS FOR USING DEVICE TO IDENTIFY, DOCUMENT, AND DIAGNOSE EYE DISEASE

(71) Applicant: Tushar Mahendra Ranchod, Oakland, CA (US)

(72) Inventor: Tushar Mahendra Ranchod, Oakland, CA (US)

(21) Appl. No.: 13/485,206

(22) Filed: May 31, 2012

Related U.S. Application Data
(60) Provisional application No. 61/491,898, filed on Jun. 1, 2011.
(60) Provisional application No. 61/501,705, filed on Jun. 27, 2011.

(51) Int. Cl. A61B 3/14

(52) U.S. Cl. CPC A61B 3/14
USPC 351/206; 351/246

(57) ABSTRACT

A device (20) for use in the screening, documentation, and diagnosis of various diseases of the eye (1). Several images (60) are taken substantially simultaneously, with the images (60) preferably taken in a non-coplanar orientation relative to each other (60). The images (60) represent multiple different zones (11) of the retina (10) taken using different optical imaging pathways (200). Image (60) distortion is minimized, because the individual optical imaging pathways (200) need to account for significantly less differential curvature of the object plane than a wide-field optical pathway that attempts to capture both the central and peripheral retina (10) in a single image. A single composite wide field image (61) can be generated, by merging the overlapping fields of multiple, concurrently captured images (60) taken at different angles.

### RELATED PATENT APPLICATIONS

This patent application claims the benefit of commonly-owned U.S. provisional patent applications 61/491,898 filed Jun. 1, 2011, and 61/501,705 filed Jun. 27, 2011, which two provisional patent applications are hereby incorporated by reference in their entireties into the present patent application.

### TECHNICAL FIELD

This invention pertains to the field of examining the eye, and obtaining therefrom relevant diagnostic information.

### BACKGROUND ART

Retinal imaging is commonly used both to screen for retinal diseases and to document findings observed during clinical examination. Retinal photography (sometimes referred to as fundus photography) is presently performed in a variety of ways. In ophthalmology and optometry clinics, fundus photographs made with cooperative adult patients are usually taken with a non-contact digital fundus camera. The camera does not come into direct contact with the eye 1. Fundus cameras may use wide field optics to capture a wide field view in a single image, or they may use sequential multiple, partially overlapping, images to create a composite wide field image. In neonates and children who require retinal imaging, digital photographs are most commonly taken with a contact camera system in which a camera hand piece is placed directly against the ocular surface 4 after topical anesthesia is administered. Such systems can be used during examination under anesthesia when the child is asleep, or with an awake child if the child is cooperative or too small to resist enforced positioning.

Digital fundus photography is increasingly being used to screen for diseases such as retinopathy of prematurity (ROP) in neonates and diabetic retinopathy in adults. In neonates, several studies have validated the use and efficacy of screening photographs in place of bedside examination by an ophthalmologist trained in ROP evaluation, as long as an ophthalmologist is available to perform bedside examination if screening photographs suggest pathologic changes requiring further evaluation.

When telemedicine screening is performed, a standard set of images is taken and transmitted to a trained reader for evaluation. The image set generally includes an external photograph, a retinal image centered on either the optic nerve 7 or macula 8 (the central retina 10), and four mid-peripheral retinal images centered superior, inferior, nasal, and temporal, respectively, to the disc and macula 8. Because these images are taken sequentially over a finite amount of time, the relative positioning of the photographs is unpredictable, and fine scale maneuvering to obtain more precisely centered images requires a longer imaging session.

Contact retinal imaging systems do not currently provide true wide field images, where “wide field” is defined as an equator-to-equator view in a single image. This is a disadvantage of present retinal imaging systems, as wide-field images have advantages compared to standard-field images for diagnosis of some medical conditions or eye 1 diseases. Vision-threatening retinal pathology is often located in the mid-periphery or far periphery of the retina 10, and may be difficult or impossible to view with standard-field imaging. Also, with standard-field imaging, multiple images are required to comprehensively image both the central and peripheral retina 10, and the acquisition of multiple images per eye 1 involves more time and patient discomfort than a single image.

Some non-contact retinal imaging systems have been able to achieve wide-field images, but these systems typically produce images with significant optical distortion towards the edges of the field. Because mid-peripheral or peripheral retinal pathology may be relatively subtle and challenging to detect without high quality images, peripheral image distortion may prevent detection or documentation of vision-threatening pathology. Even when peripheral pathology can be successfully imaged, peripheral distortion may prevent accurate evaluation of lesion size, dimensions, or other characteristics.

What is desired are systems, devices, and methods for creating images for use in detecting, documenting, and diagnosing retinal conditions or disease that overcome the disadvantages of conventional approaches, particularly with regards to obtaining wide-field images for use in diagnosing instances of mid-peripheral or peripheral retinal pathology.

### DISCLOSURE OF INVENTION

Embodiments of this invention relate to a retinal imaging device 20 that can be used to identify, document, and/or diagnose diseases of the eye 1. In some embodiments, the invention includes multiple optical lenses 210, 211 within optical pathways 200 arranged at different angles relative to the retina 10, and relative to each other, which are used to obtain multiple images 60. These individual images 60 are then combined to create a composite, wide field retinal image 61. As used herein, “image” means a still image (i.e., photograph) or a moving image (i.e., video).

In some embodiments, the multiple optical pathways 200 are interspersed with one or more non-coaxial illumination sources 300 to provide illumination for imaging (where “non-coaxial” refers to the beam generated by the source 300 being oriented so that it is not aligned with any of the optical paths 21 of the pathways 200). This provides broad retinal illumination with minimal light artifacts, because use of non-coaxial lighting 300 reduces central light-induced artifacts in the resulting images 60. The light sources 300 can comprise light emitting diodes (LEDs).

In some embodiments, the inventive system is used to create a single, focused, composite wide field image 61 using multiple partially overlapping digital images 60 of the retina 10 taken concurrently or in rapid sequence using the inventive device 20.

Embodiments of the invention described herein provide a significant improvement in contact retinal imaging systems and devices. In some embodiments, the invention produces an array of images 60 taken substantially simultaneously, with the images 60 being in a non-coplanar orientation relative to each other (i.e., the plurality of images 60 are such that they provide views along non-coplanar paths 21). Since the retina 10 has a concave curvature due to its position inside a spherical structure (the eyeball 1), embodiments of this invention permit multiple and different zones 11 of the retina 10 to be imaged using different optical pathways 200 that are non-coplanar with respect to each other 200. Image 60 distortion is lessened with the inventive approach, because the optical pathway 200 needs to account for significantly less differential curvature of the object plane than does a prior art wide field optical pathway that attempts to capture both the central and peripheral retina 10 in a single image.

Because the retina 10 must be adequately illuminated by an external light source in order to capture a suitable image, some existing systems that rely on a single optical pathway use a “donut” illumination system in which a continuous or near-continuous circle of illumination is placed around the imaging path. This “donut” type of illumination source avoids coaxial placement of the imaging pathway and illumination pathway, and thereby avoids a central light reflection artifact in the captured image. However, a disadvantage is that such a system introduces a circular reflection artifact.

In contrast, embodiments of the present invention take advantage of multiple illumination sources 300 distributed among or between the multiple optical pathways 200. At least one of the illumination sources 300 is not coaxial with any of the multiple optical pathways 200 (and typically none 300 are), so that the captured images 60 are not marred by a central light reflex. Because the multiple captured images 60 are partially overlapping, and the relative positions and angles of the images 60 are known, predictable non-central light reflection artifacts are removed at the time of composite image 61 generation (as long as each area of retina 10 that is masked by a light reflection artifact in one image 60 is imaged without a reflection artifact by another image 60 due to the partial overlapping of the multiple generated images 60). Although there are some variations in ocular structure among patients (such as axial length, corneal curvature, and total refractive error), the approximate position of light reflection artifacts generated when using embodiments of this invention is predictable; thus, the multiple optical imaging pathways 200 and multiple illumination sources 300 can be arranged to place the reflection artifacts in such a way as to ensure that partial image 60 overlap provides at least one clear image 60 of every zone 11 within the larger composite image 61.

According to one embodiment, this invention includes a device 20 operative to provide a retinal image 60 by generating multiple digital photographs of the retina 10, with the images 60 being taken at different angles through the pupil 2. The device 20 may include a single chassis 100 with a smooth concave surface that fits against the ocular surface 4, with or without a viscous coupling agent, and with or without a disposable or reusable transparent cover 110 positioned between the bottom surface 101 of the chassis 100 and the ocular surface 4. Chassis 100 may or may not contain optical or structural elements critical to image 60 capture or retinal illumination. As used herein, “chassis 100” refers to any suitable structure that is able to hold the constituent items 200, 300, 220, 221, 230 in the desired configuration with respect to ocular surface 4.

Within the chassis 100 are a plurality of discrete optical imaging pathways 200, with each pathway 200 being aimed through the pupil 2 at a different angle in order to capture images 60 of different zones 11 of the retina 10. Each pathway 200 may include one or more optical lenses 210, 211 in either a fixed or variable position in order to focus an image of one retinal zone 11 onto a digital sensor 220 or portion of a common digital sensor 221. Each pathway 200 may be either substantially linear or substantially non-linear, achieved through the use of mirrors, straight or curved light pipes 30, or straight or curved fiber optic bundles 30. The chassis 100 may contain one digital sensor 220 for each discrete optical imaging pathway 200. Alternatively, multiple discrete optical imaging pathways 200 may direct different retinal images 60 onto different areas of one or more common digital sensors 221. Similarly, each discrete optical pathway 200 may contain its own set of one or more lenses 210, 211, or one or more lenses 210, 211 may be common to or shared between or among multiple discrete common pathways 200.

According to another embodiment, this invention includes a device 20 operative to generate multiple digital images 60 of the retina 10 substantially concurrently and at different angles, with one or more light sources 300 interspersed among or between multiple discrete optical imaging pathways 200 positioned within a single chassis 100. At least one of the light sources 300 is not coaxial with any of the discrete optical imaging pathways 200. Light sources 300 may be located substantially adjacent to the chassis 100 surface 101 that comes in contact with the ocular surface 4 (see FIG. 3), or light sources 300 may be located distal from said chassis 100 surface 101 (see FIGS. 5 and 6), in which case light pipes or fiber optic bundles may be used to transmit light from the light source 300 to said chassis 100 surface 101. The light sources 300 are preferably separated from the optical imaging pathways 200 by substantially opaque dividers 230, with the opaque dividers 230 extending to the outermost chassis surface 101. Each illumination transmission point at the chassis 100 surface 101 may correspond to a separate distal illumination source 300, or multiple illumination transmission points at the chassis 100 surface 101 may correspond to a single distal illumination source 300 within the chassis 100.

The set of light sources 300 may comprise means to provide varying intensity of light and/or different wavelengths of light. For example, sources 300 may comprise one or more white light sources 300 and one or more blue light sources 300, which may be utilized at different times or in combination to more effectively image different structures or elements within the eye 1. While retinal images 60 are frequently captured using the entire visible light spectrum, certain features such as blood vessels and vascular abnormalities may be better seen with illumination in a limited light spectrum. Many retinal diseases require fluorescein or indocyanine green angiography for accurate diagnosis, and these imaging modalities typically utilize specific light filters on both the light source and the image capture sensor. Accordingly, the illumination sources 300 and/or the optical imaging pathways 200 of the present invention may utilize one or more light filters to limit the spectrum of illumination or image 60 capture.

In some embodiments, the imaging pathways 200 contain a plurality of lens 210, 211 and/or sensor 220, 221 sub-sections, each of which is used to capture and detect light in a portion of the spectrum. If different light spectra are captured separately at the level of the digital sensors 220, 221, various image 60 types (full color; red-free; angiography-appropriate filtered) can be composed from these separately captured spectra and used for imaging the variety of structures within the eye 1 (some of which, as mentioned, may best be observed at specific wavelengths or in the absence of specific wavelengths). Alternatively, if full color images 60 are captured at the level of the sensor 220, 221, various image 60 types can be produced by a combination of hardware, firmware, and/or software after image 60 capture takes place.

According to another embodiment, this invention includes a system 20, 40 operative to generate multiple, partially overlapping digital retinal images 60 for use in creating a single composite retinal photograph 61 with a field of view wider than any one of the individual images 60. The composite digital image 61 (still or video) is generated by combining multiple, partially overlapping concurrent images 60. The composite images 61 may be used for color fundus imaging, red-free fundus imaging, angiography with intravenous administration of a dye such as fluorescein or indocyanine green, or other visualization requiring discrete light spectra for illumination and/or image capture. It is noted that while current retinal imaging systems may allow for manual or semi-automated creation of composite images, embodiments of this invention permit fully automated creation of composite images 61 using a combination of hardware, firmware, and/or software, because the relative positions and angles of the multiple discrete retinal images 60 (as well as possible light reflection artifacts) are known and can be accounted for when processing the individual images 60 into the composite image 61.

### BRIEF DESCRIPTION OF THE DRAWINGS

These and other more detailed and specific objects and features of the present invention are more fully disclosed in the following specification, reference being had to the accompanying drawings, in which:

FIG. 1 is a prior art side (transverse) cross-sectional view of a human eye 1.

FIG. 2 is a prior art planar view, taken along view lines 2-2 of FIG. 1, of retina 10.

FIG. 3 is a side (transverse) cross-sectional view of the eye 1 showing the imaging device 20 of the present invention.

FIG. 4 is a side (transverse) cross-sectional view of a first alternative embodiment of the device 20 of FIG. 3, in which prisms 240 are used.

FIG. 5 is a side (transverse) cross-sectional view of a second alternative embodiment of the device 20 of FIG. 3, showing light pipes/fiber optic bundles 30.

FIG. 6 is a side (transverse) cross-sectional view of the FIG. 5 embodiment showing paths 39 of illumination sources 300.

FIG. 7 is a side (transverse) cross-sectional view of the eye 1 of FIG. 1, showing three retinal zones 11.

FIG. 8 is a planar view, taken along view lines 8-8 of

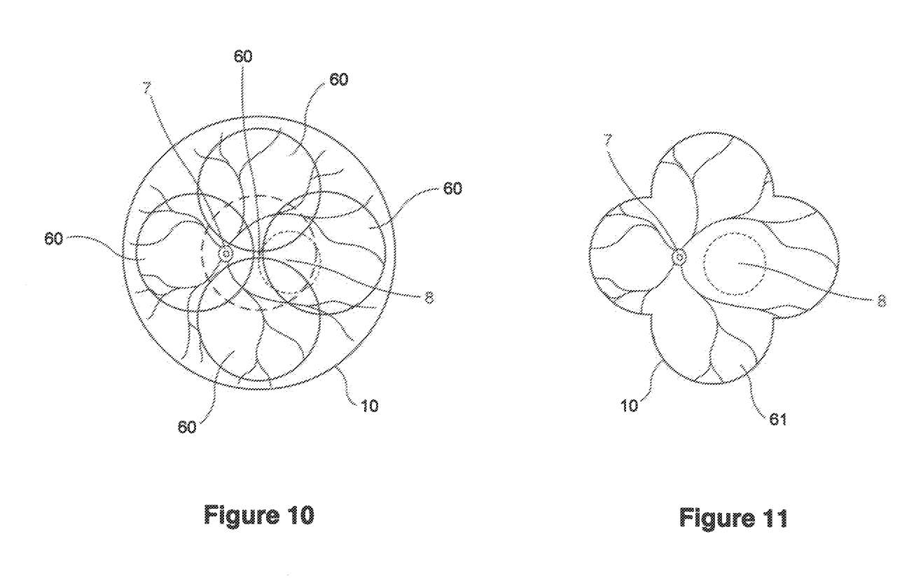

FIG. 7, showing the three retinal zones 11.

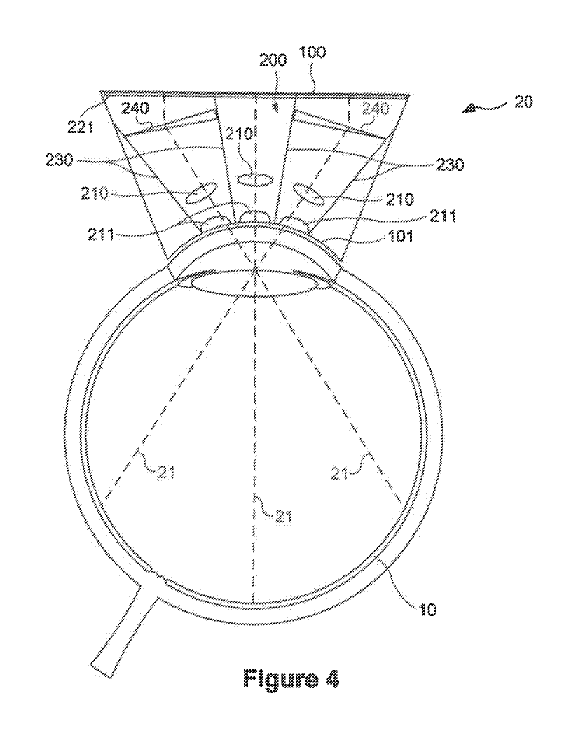

FIG. 9 is a bottom planar view, taken along views 9-9 of FIG. 3 and through transparent cover 110, showing the arrangement of optical pathways 200 and light sources 300.

FIG. 10 is a planar view of retina 10 being imaged by a device 20 having the features of FIG. 9.

FIG. 11 is a planar view of retina 10 showing a wide field composite image 61 generated by combining the individual images 60 of FIG. 10.

FIG. 12 is a sketch of a control unit 40 coupled to device 20.

Note that the drawings are not rendered to any particular scale or proportion.

### DETAILED DESCRIPTION OF PREFERRED EMBODIMENTS

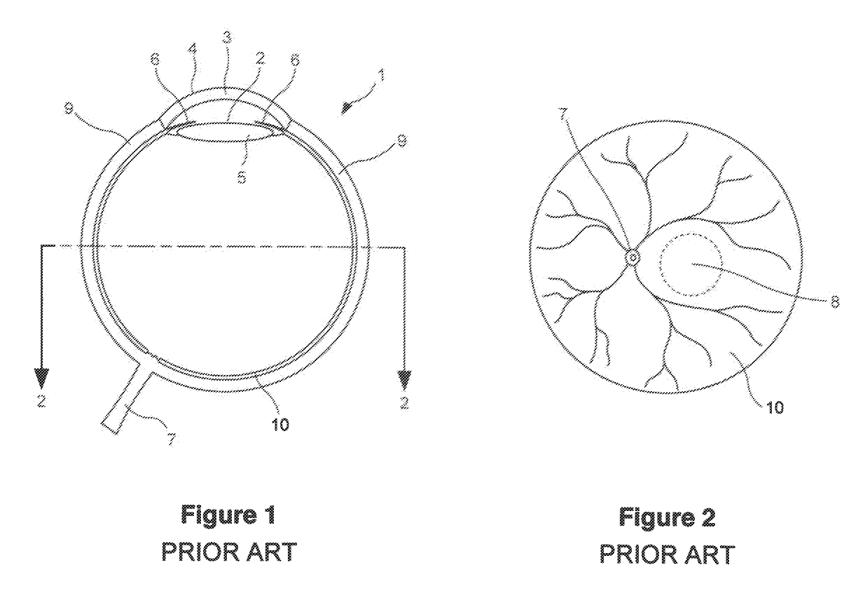

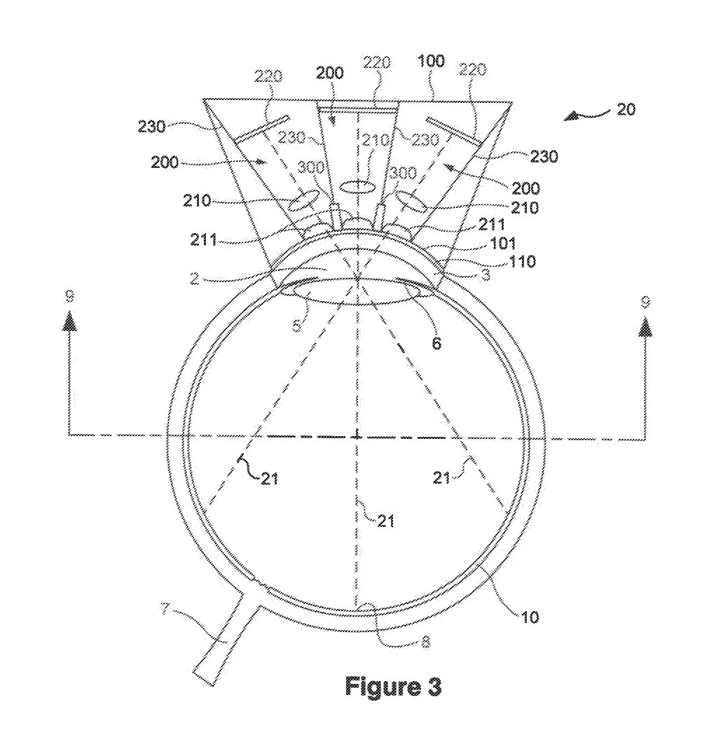

FIG. 1 illustrates the basic anatomy of the human eye 1 in transverse cross section. FIG. 2 illustrates corresponding landmarks on the retina 10. Structures of the eye 1 that will be referred to in describing one or more embodiments of this invention include: cornea 3, sclera 9, corneoscleral ocular surface 4, which may be placed in physical contact with one surface 101 of chassis 100; iris 6, pupil 2, through which optical paths 21 pass in order to capture images 60 of the retina 10; lens 5, through which optical paths 21 pass in order to capture images 60 of the retina 10; and the retina 10 itself, which is imaged using embodiments of the invention, and which is centered at the macula 8. The cornea 3, sclera 9, and adjacent tissues constitute the ocular surface 4 against which device 20 may be placed in direct contact, with or without a disposable or reusable transparent cover 110.

The average corneal diameter in a newborn human is approximately 9-10 mm and in an adult human is approximately 12 mm, but may be lesser or greater for any given individual. The internal optics of the human eye 1 are determined primarily by the curvature of the cornea 3 and lens 5, in conjunction with the axial (front to back) length of the eye 1. The average axial length of a newborn human eye 1 is approximately 18 mm and in an adult human eye 1 is approximately 24 mm, but may be lesser or greater for any given individual.

FIG. 3 illustrates a side view of an exemplary embodiment 20 of the invention, in which chassis 100 is placed against the ocular surface 4, with the chassis 100 containing multiple, discrete optical imaging pathways 200 aimed through the pupil 2 at different angles to image different zones 11 of the retina 10 (eee FIG. 7). The chassis 100 and optical imaging pathways 200 share a common interface which approximately matches the curvature of the human cornea 3 and/or sclera 9. Each discrete optical imaging pathway 200 has an optical path 21 aimed through the pupil 2 and lens 5 towards a zone 11 of the retina 10. The optical paths 21 define the direction along which the images 60 are obtained; the center lines of paths 21 are shown as dashed lines in FIGS. 3, 4, and 5.

The discrete optical imaging pathways 200 may be adjacent to each other as shown in FIG. 3, or they may be separated from each other. Each pathway 200 is preferably surrounded by opaque dividers 230, which function to reduce light from passing into or out of that pathway 200 except through transparent cover 110, which is situated at the bottom (outermost) end 101 of chassis 100.

Each optical imaging pathway 200 is substantially continuous with the chassis 100 surface 101 that touches the ocular surface 4, with such contact being either directly or indirectly through disposable or reusable transparent cover 110. In the embodiments shown, each pathway 200 is non-coplanar with all other pathways 200, although in some embodiments, it is sufficient that two or more pathways 200 are coplanar.

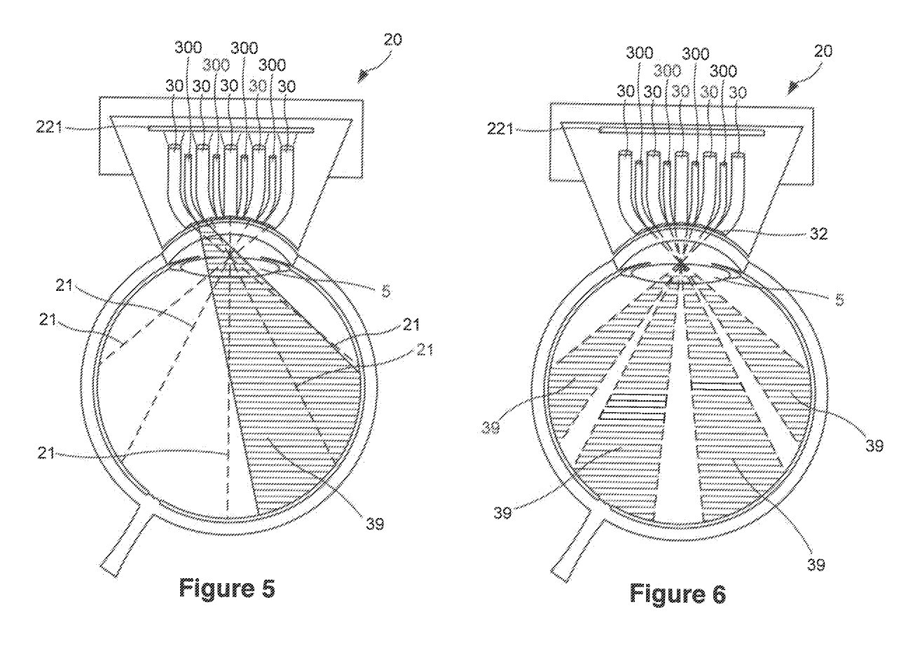

In some embodiments, each optical imaging pathway 200 is arranged approximately, though not necessarily exactly, perpendicular to the external corneal surface 4, or approximately at an angle aimed from a particular spot on the corneal surface 4 towards the pupillary aperture 2. Each optical imaging pathway 200 typically contains one or more optical lenses 210, 211 placed in such a way that the lens system 210, 211 directs an image 60 of the corresponding retinal zone 11 onto a dedicated digital sensor 220 or part of a common digital sensor 221 (see FIGS. 4 through 6). It may well be desirable to focus the images within pathways 200, given that different eyes 1 have different focal points. Focusing can be accomplished using light field imaging (for example, cameras that use an array of fish-eye lenses in front of the sensor to allow for post-hoc change of composite image focal plane). Another way to accomplish focusing is to configure chassis 100 to have a long depth of field, with one or more different versions of a disposable tip that can fit on the bottom 101 of chassis 100 with some focal power. For example, one version of the tip can be suitable for a pediatric or small adult eye 1, a second version of the tip can be suitable for a normal adult eye 1, and a third version of the tip can be suitable for an adult large/myopic eye.

The expression “digital sensor” 220, 221 encompasses a flat or concave digital sensor as well as the accompanying interconnections, power supply, and hardware, firmware, and/or software needed for image 60 processing and output. Similarly, the illustrated chassis 100 is a simplified illustration and does not show the interconnections, power source, and/or attachments necessary for the device 20 to function as intended.

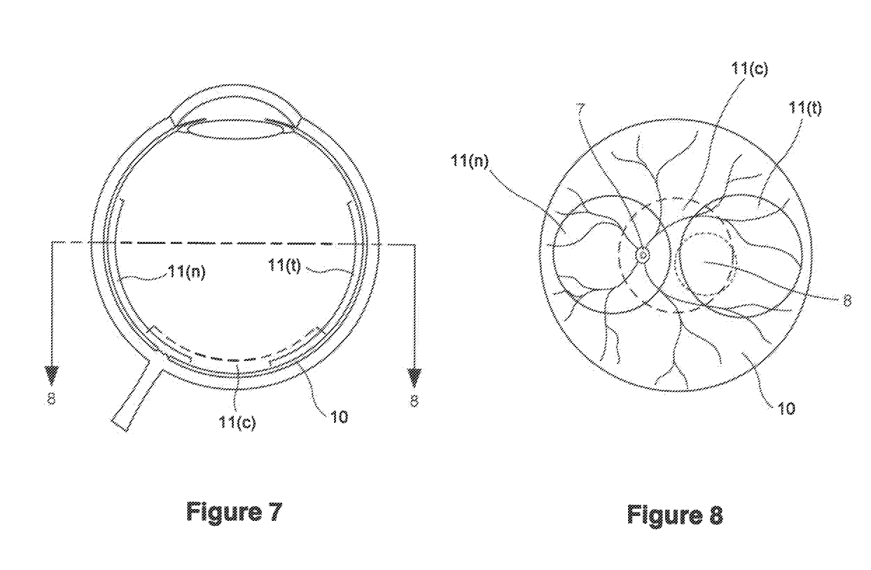

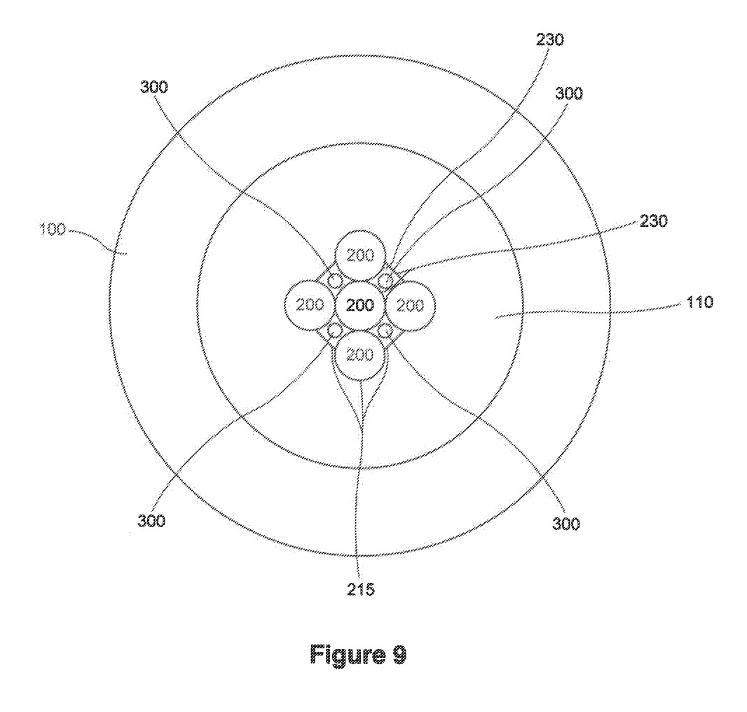

Each individual illumination source 300 may be collimated and aimed directly at the retina 10, or may be focused by one or more optical lenses (not illustrated), typically located near the chassis 100 outer surface 101 to limit the diameter of the illumination core 39 as it crosses the lens 5 (see FIGS. 5 and 6). At least one of the light sources 300 is preferably non-coaxial with respect to all of the pathways 200. Each illumination source 300 may be directed to one or more regions 11 of the retina 10 by way of light pipes, fiber optic bundles, beam splitters, or lenses, which may constitute either linear or non-linear optical pathways.

As used herein, “illumination source 300” encompasses the power supply and interconnections necessary to operate the source 300. Although each illumination source 300 is depicted in FIG. 3 as containing one bulb, LED, or other light source, each source 300 may contain multiple illumination sub-sources having the same or different characteristics (e.g., different intensity or different emitted spectrum of light). Variable intensity of illumination may be desirable, because greater light intensity reduces patient comfort during imaging, and one goal of imaging may be to obtain usable images 60 at the lowest possible illumination intensity. A variable emitted spectrum of light for the light sources 300 may be desirable, because certain procedures (such as fluorescein angiography and indocyanine green angiography) require specific light emission spectra from the illumination source 300 to be used, in conjunction with image capture filters with different specific light spectra.

Fluorescein angiography is a common type of diagnostic technique used in ophthalmology, in which the retina 10 is illuminated with a 490 nm bandpass filtered blue light, and the sensor captures only 520 nm to 530 nm bandpass filtered yellow-green light. Use of illumination filters can entail device 20 having a second set of illumination sources 300 (one with white light and one with a 490 nm output). Alternatively, chassis 100 can have a unique disposable tip that, instead of being clear (for color photography), has colored filters built into it (potentially separate filters for illumination and for imaging). Alternatively, the digital sensor(s) 220, 221 can be programmed by software to process only specific wavelengths, or the digital sensors 220, 221 may contain multiple discrete subsensors that process different wavelengths, so filters over the imaging pathways 200 may or may not be necessary.

Each device 20 may optionally comprise a disposable clear (plastic or equivalent) tip that is single use for each patient. The disposable tip may or may not have optical power that relates to either the illumination or imaging aspects of the device 20. The tip can be clear or contain color filters for angiography. Also, chassis 100 can comprise different tips with different optical power for different eyes 1, e.g., a pediatric tip (for small, pediatric, or hyperopic eyes 1), a normal adult tip, and a large adult tip (for very long or myopic eyes 1).

Focusing may be achieved in one of several ways: 1) moving the lenses 210, 211 by servos; 2) moving the lenses 210, 211 by a manual mechanism (like a traditional camera zoom lens, for example); 3) light field imaging using fish-eye lens arrays and post-hoc software reconstruction (like the Lytro and Pelican cell phones and DSLR cameras, respectively); or 4) using a high depth of field combined with different disposable tips having different optical powers.

The device 20 depicted in FIG. 3 is configured to have a different sensor 220 corresponding to each pathway 200, while the devices 20 depicted in FIGS. 4 through 6 are configured to have a single common sensor 221 corresponding to all discrete optical imaging pathways 200. In general, a common sensor 221 can be used with any two or more pathways 200.

The device 20 depicted in FIG. 4 uses prisms 240 to create parallel beams 21 of light at the sensor 221 surface. The prisms 240 correct for the angle of difference between the optical path 21 of any given pathway 200 and the surface of the common digital sensor 221. Prismatic or other optical or chromatic distortion at the plane of the sensor 221 may be adjusted using any combination of hardware, firmware, software and/or additional optical lenses.

The device 20 depicted in FIGS. 5 and 6 is configured to have multiple linear and/or non-linear optical imaging pathways 200 utilizing fiber optic bundles 30 or light pipes 30 to obtain images 60 at different angles through the pupil 2, and to deliver multiple of these images 60 in parallel at the common sensor plane 221.

FIG. 5 depicts an embodiment in which the illumination cones 39 are directed at the retina 10 without an attempt to minimize the diameters of the cones 39, while FIG. 6 depicts an embodiment of the invention in which the illumination cones 39 are focused to minimize the diameters of the cones 39 as they pass through lens 5. In FIG. 5, just one cone 39 is shown, to avoid cluttering the drawing. For the same reason, imaging paths 21 are not shown in FIG. 6.

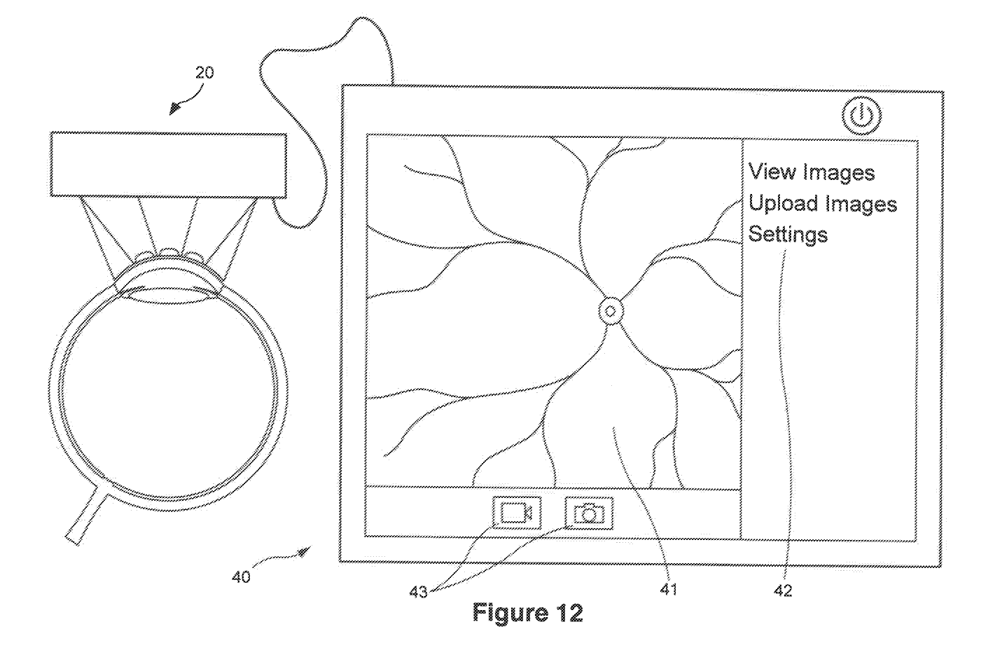

The optical paths 21 corresponding to the pathways 200 may be arranged to capture images 60 of certain retinal zones 11 from multiple and significantly different angles, e.g., in order to obtain stereoscopic image pairs of anatomic landmarks, such as the optic nerve head 7 or macula 8. A retinal zone 11 is defined as an area of retina 10 corresponding to a circumscribed object plane or part of a circumscribed object plane which has one or more image planes corresponding to an optical imaging pathway 200. Three such partially overlapping zones 11 are depicted in FIGS. 7 and 8: nasal, central, and temporal. These three zones 11 have been imaged by three discrete optical imaging pathways 200.

The number and positioning of retinal zones 11 and corresponding discrete optical imaging pathways 200 may vary based on lens 210, 211 characteristics, such as the diameter and spacing of the lenses 211 most proximal to the corneal surface 4. For example, an 8 mm diameter central zone 11(c) of the cornea 3 might accommodate a 3-lens-across array of lenses 211 that are each 2 mm in diameter, or a 5-lens-across array of lenses 211 that are each 1 mm in diameter. The usable central corneal area for such an array depends on the degree of dilation of the pupil 2; a greater corneal area can be utilized if a greater degree of pupil 2 dilation is assumed or required for use of the device 20. A greater degree of dilation beyond the minimum required will have minimal impact on image 60 acquisition and image 60, 61 quality, whereas a lesser degree of pupil 2 dilation may reduce image 60, 61 quality due to glare from iris 6 illumination. The peripheral extent of the composite image 61 may be reduced due to blockage of optical pathways 200 by the iris 6.

In the embodiment illustrated in FIG. 9, the discrete optical imaging pathways 200 are placed as shown in FIG. 3, although other placements or arrangements may be used without departing from the concepts underlying this invention. In the FIG. 9 embodiment, the light sources 300 are dispersed among the optical imaging pathways 200, and the light sources 300 and optical imaging pathways 200 are separated from each other by opaque dividers 230 which function to limit the reflection and transmission of light. Different numbers and distributions of optical imaging pathways 200 and light sources 300 are possible. Preferably, at least one illumination source 300 is non-coaxial with respect to all of the optical imaging pathways 200, so as to reduce reflective artifacts from the light sources 300 in the digital images 60 obtained by the device 20. The light sources 300 are preferably distributed among imaging pathways 200 in a non-linear manner, and they may be distributed primarily within the outer boundary 215 of the optical imaging pathways 200, rather than being arranged outside of boundary 215 in a circumferential manner.

The relative focal points of the optical imaging pathways 200 may be fixed, or they may vary with respect to one another according to a predefined algorithm, in order to produce multiple, partially overlapping retinal photographs 60 which have a sufficiently low degree of optical distortion at their edges.

FIG. 10 depicts an exemplary representation of the retina 10 in which five different, but partially overlapping, zones 11 of the retina 10 are imaged. FIG. 11 shows these five individual images 60 having been merged to create a single composite wide field retinal image 61. The creation of the composite image 61 can be produced through any suitable combination of hardware, firmware, and/or software (not illustrated), either immediately following image 60 acquisition or at a later time. The merging of the images 60 may be easily automated, because the hardware/firmware/software imaging system is not required to determine the approximate relative positions of the images 60. Instead, it has to make just relatively fine adjustments to create the composite image 61.

The algorithm used to create composite retinal image 61 from multiple, partially overlapping retinal images 60 may be identical or similar to currently available photographic “stitching” algorithms, whereby software identifies common elements across overlapping images 60 in order to match the overlapping zones of adjacent images 60 to create the composite image 61. An important difference when constructing a composite image 61 using the invention, however, is the fact that the relative angles and positions of adjacent images 60 are already known, since the relative angles of the multiple discrete optical pathways 200 are fixed. Consequently, the composite images 61 can be generated with greater accuracy and greater precision than existing methods that rely on assumptions of relative positioning or complex inputs from the user to guide the software in making decisions about overlapping image zones 11.

Note that images obtained through the edges of optical lenses tend to be more distorted than images obtained through the centers of optical lenses. This invention's use of multiple optical imaging pathways 200 allows for a significant amount (if not most) of the imaged area of retina 10 to be captured through the center or mid-periphery of the various lenses 210, 211. In contrast, a widefield image of the retina 10 obtained through a single optical imaging pathway using a prior art device necessarily captures the periphery of the retina 10 through the periphery of one or more lenses, with consequent image distortion, which may render the obtained image unsatisfactory for purposes of diagnosing or treating certain diseases of the eye 1.

FIG. 12 shows device 20 being coupled to a control unit 40 by wire, but the coupling could be done wirelessly or the control unit 40 may be built into either the same chassis 100 as device 20 or integrated into a computer or mobile device. Control unit 40 comprises a digital computer, a touch or non-touch display 41, and a user interface 42, which may consist of touch screen inputs or other means by which a user can communicate with control unit 40, such as via a keyboard or mouse. At the bottom of display 41 are two mode buttons 43, represented by icons for a video imaging mode and a still imaging mode, respectively.

Image 60, 61 display on a digital screen could be built into chassis 100, but more typically is contained in separate control/display unit 40. Display 41 allows the user to view images 60, 61. User interface 42 comprises control functions for device 20 (shoot, focus, illumination intensity, etc.). Thus, control unit 40 allows the user to transmit command data to device 20, as well as to receive output from device 20. Command inputs may also be built into the chassis 100 of the device 20.

The hardware, firmware, and/or software that produces the composite image 61 from the individual images 60 is typically located within control unit 40. When implemented in software, the software instructions for generating the composite images 61 may be wholly or partially implemented in the form of a set of instructions executed by a central processing unit (CPU) or microprocessor contained within control unit 40. The instructions may be stored in a memory or other data storage device, embedded on one or more computer readable media, or provided in any other suitable manner.

The multiple original partially overlapping retinal images 60 typically remain available for display on display 41 for user review, along with the composite retinal image 61.

Among others, uses of the invention for screening, diagnosis, or documentation of ocular disease include:
1) Screening for retinopathy of prematurity (ROP). Extremely premature neonates are at risk of developing ROP, a disease which may cause blindness if not diagnosed and treated in a timely manner. The American Academy of Pediatrics and the American Academy of Ophthalmology have published guidelines regarding the appropriate timing for evaluation for ROP by ophthalmologists trained specifically in the evaluation of ROP. Local telemedicine, in which a local certified ophthalmologist remotely reads retinal images taken by non-ophthalmic staff in a neonatal intensive care unit, is a well validated paradigm. Patients with abnormalities on imaging warrant clinical examination at the bedside, while patients with normal retinal images 60 may be followed serially with further imaging. ROP telemedicine screening requires the acquisition of a standard set of images, since a single image cannot currently capture an adequately wide field of view to detect ROP with adequate sensitivity. Embodiments of the invention permit rapid acquisition of a wide-field retinal image 61, with image 60 acquisition that has potentially higher resolution and using a procedure that is less uncomfortable for the patient when compared to existing contact retinal imaging systems.
2) Retinoblastoma is the most common primary intraocular malignancy in humans. Retinoblastoma is most frequently diagnosed in newborns or young children, and threatens vision, and potentially life, depending on disease severity at the time of diagnosis. Patients diagnosed with retinoblastoma currently are required to undergo contact retinal imaging on a regular basis in order to document the appearance of the retina 10 and tumor before and after treatment. Use of this invention permits rapid and simple acquisition of such images 60, and due to its small form factor and ease of use, the invention also potentially allows for screening of newborns or infants for blinding retinal diseases such as retinoblastoma.
3) Hospitalized patients diagnosed with Candidemia, a type of fungal bloodstream infection, are routinely evaluated by an ophthalmologist for evidence of chorioretinal involvement of the infection. Ocular involvement of Candida infection may alter the duration or intensity of treatment with intravenous or oral antifungal medications, and ocular involvement may also be used to monitor response to treatment at a systemic level. This invention may be used for local or telemedicine screening of patients with Candidemia by a local ophthalmologist. The images 60 may be acquired and interpreted locally by an ophthalmologist. Alternatively, the images 60 can be acquired by an intensive care unit or other hospital unit staff, and read and interpreted by a local ophthalmologist, with the results sent back to the ordering physician to incorporate into the diagnosis and treatment plan. Normal images 60, 61 would not warrant bedside examination, whereas abnormal images 60, 61 may warrant a bedside examination by a local ophthalmologist.
4) Newborns seen by a pediatrician or pediatric subspecialist (such as a neonatologist) may be appropriate for retinal photographic screening based on risk of retinal pathology or the desire of the patient's guardian. The invention can be used by a pediatrician or other health care provider to image the retina 10 and obtain an interpretation of the image 60, 61 either locally by a device such as control unit 40 or using a telemedicine infrastructure.

While certain exemplary embodiments have been described in detail and shown in the accompanying drawings, it is to be understood that such embodiments are merely illustrative of, and not intended to be restrictive of, the broad invention, and that this invention is not to be limited to the specific arrangements and constructions shown and described, since various other modifications may occur to those with ordinary skill in the art. For example, this invention can be used to image eyes other than human eyes, such as the eyes of non-human animals. As another example, the plurality of discrete optical imaging pathways 200 can be arranged in a manner to enable capture of stereoscopic pairs of images 60 of one or more retinal zones 11. This embodiment allows for three-dimensional image 60 viewing of either discrete retinal zones 11 or the entire area included in the composite retinal image 61.

What is claimed is:

1. A device for imaging an eye, said device comprising:
a plurality of optical pathways, each pathway oriented to image a particular area of the eye;
coupled to each pathway, a sensor for capturing image information corresponding to that pathway; and
coupled to the sensors, means for generating individual images of the areas of the eye corresponding to the pathways.

2. The device of claim 1 wherein each image is a still image.

3. The device of claim 1 wherein each image is a video image.

4. The device of claim 1 further comprising, coupled to the generating means, means for fabricating a composite image from the individual images.

5. The device of claim 4 wherein the fabricating means is fully automated.

6. The device of claim 1 wherein no two pathways lie in the same plane.

7. The device of claim 1 further comprising a plurality of illumination sources interspersed among the pathways, wherein at least one of the illumination sources is non-coaxial with respect to all of the pathways.

8. The device of claim 7 wherein at least one illumination source is adjustable with respect to at least one of intensity and wavelength.

9. The device of claim 1 wherein at least one pathway comprises at least one lens.

10. The device of claim 1 wherein the pathways are separated by opaque dividers.

11. The device of claim 1 wherein at least two pathways share a common sensor.

12. The device of claim 1 wherein at least one pathway comprises a prism.

13. The device of claim 1 wherein at least one pathway comprises a fiber optic bundle or a light pipe.

14. The device of claim 1 wherein the pathways are contained within a single chassis; and
the chassis is optically coupled to the eye via a disposable or reusable transparent cover.

15. The device of claim 1 comprising:
a chassis containing the pathways and the sensors; and
coupled to the chassis, a control unit comprising a display for viewing the images, said control unit adapted to convey commands to the chassis.

16. A method for imaging an eye, said method comprising the steps of:
simultaneously focusing a plurality of optical pathways on different areas of the eye;
capturing imaging information corresponding to each pathway; and
gathering the imaging information from the pathways to generate a plurality of individual images, each individual image corresponding to the area corresponding to a given pathway.

17. The method of claim 16 wherein the areas being imaged comprise zones of the retina.

18. The method of claim 16 wherein the imaging is used to detect at least one of ROP, retinoblastoma, and Candidemia.

19. The method of claim 16 wherein at least two of the areas being imaged comprise a stereoscopic image pair of an area of the retina.

20. The method of claim 16 further comprising the step of combining the individual images together to form a composite image of a wide field of the eye.
